(19) United States
(12) Patent Application Publication
(10) Pub. No.: US 2024/0316197 A1
(43) Pub. Date: Sep. 26, 2024

(54) HUMAN IPSC-DERIVED MACROPHAGE

(71) Applicant: The Regents of the University of California, Oakland, CA (US)

(72) Inventors: Dan S. Kaufman, San Diego, CA (US); Somayeh Pouyanfard, San Diego, CA (US)

(21) Appl. No.: 18/579,425

(22) PCT Filed: Jul. 15, 2022
(86) PCT No.: PCT/US2022/037303

§ 371 Date: Jan. 15, 2024

Related U.S. Application Data
(60) Provisional application No. 63/221,994, filed on Jul. 15, 2021.

(51) Int. Cl. A61K 39/00; A61K 35/15; A61K 35/545; A61P 35/00; C07K 16/28

(52) U.S. Cl. CPC A61K 39/4614; A61K 35/15; A61K 35/545; A61K 39/4631; A61K 39/464462; A61K 39/464466; A61P 35/00; C07K 16/2803; C07K 16/2863; A61K 2039/505; A61K 2239/59

(57) ABSTRACT

Human iPSC-derived macrophages, methods for the manufacture thereof, and methods of treatment of cancer and other conditions therewith. Human iPSC-derived macrophages further comprise a chimeric antigenic receptor (CAR) expressed thereon, such as Bai1, MegF10 or MerTK, referred to as iPSC-derived CAR-expressing macrophages (iPSC-CARMAs). The methods of treatment provide further co-administering to the subject an effective amount of iPSC-CARMAs and an antibody specific for the cancer, such as anti-CD47 or anti-EGFR antibody. The iPSC-derived macrophages promote phagocytic activity, reduce tumor burden, and improve subject survival.

### CROSS-REFERENCE TO RELATED APPLICATIONS

This application claims priority to U.S. Provisional Patent Application Ser. No. 63/221,994 filed Jul. 15, 2021, the entire contents of which are incorporated herein by reference.

### TECHNICAL FIELD

The present invention relates to engineered immunotherapies.

### BACKGROUND

Adoptive cell therapy for treatment of refractory malignancies has been gaining considerable interest in the past few years. Chimeric antigen receptor (CAR) expressing T cells have demonstrated strong efficacy against B cell malignancies, with 2 different anti-CD19 CAR-T cell products now approved by the US FDA. Natural killer (NK) cells have also been gaining increased interest. NK cells offer an advantage in that they normally function as allogeneic immune cells, so do not have to be derived or isolated on a patient specific basis. This has enabled human pluripotent stem cells, both human embryonic stem cells (hESCs) and induced pluripotent stem cells (iPSCs) to serve as an effective platform for derivation of NK cells with phenotype and gene expression profiles very similar to NK cells isolated from peripheral blood.

Additionally, hESCs and iPSCs serve as an excellent platform for cellular engineering to enhance their anti-tumor activity. Specifically, iPSC-derived CAR-expressing NK cells with NK cell-specific signaling domains have been produced that demonstrate improved targeted killing of both hematologic malignancies (anti-CD19) and solid tumors (anti mesothelin) both in vitro and in vivo. Indeed, this strategy and other iPSC-NK cell products have now entered clinical trials.

While use of CAR-T cells and CAR-NK cells have gained interest, the potency of these engineered lymphocytes for treatment of refractory solid tumors remains unclear. In large part this is due to the need for these cells to penetrate into the tumor that consists not only of the malignant cells, but also the immune suppressive tumor microenvironment (TME) that can limit immune cell activity. In contrast to T cells and NK cells, macrophages are more mobile phagocytic cells that more readily infiltrate tumors (1). Indeed, the majority of immune cells in the TME of solid tumors are typically macrophages (2). In addition to their phagocytic activity, macrophages also serve as antigen presenting cells and secrete cytokines that stimulate endogenous T cell activity (3). Recent studies have expressed CARs in primary (peripheral blood) macrophages (PB-Macs) and demonstrated improved killing of ovarian cancer cells in vitro and in vivo, both by direct killing, as well as stimulation of T cell responses (4). Indeed, these CAR-macrophages have now entered clinical trials. While these advances are very interesting and important, use of PB-Macs to produce CAR-macrophages is challenging to broad scale-up of this approach, as cells would need to be engineered on a patient-specific basis. Additionally, since these studies (and initial clinical product) use a CAR with only the CD3z chain as the intracellular signaling component, they are likely not optimal to fully stimulate macrophage activity.

### SUMMARY OF THE INVENTION

The invention provides human iPSC-derived macrophages, and methods for the manufacture thereof, as described herein. In embodiments, the human iPSC-derived macrophage further comprises a chimeric antigenic receptor (CAR) expressed thereon. In embodiments, the CAR is Bai1, MegF10 or MerTK. In embodiments, the invention promotes phagocytosis of cancer cells, such as ovarian cancer cells.

The invention provides a method of treating a cancer comprising administering to a subject in need thereof an effective amount of a human iPSC-derived macrophage described herein. In embodiments, the invention provides further administering to the subject an effective amount of an antibody specific for the cancer. In embodiments, the antibody is an anti-CD47 or anti-EGFR antibody. In embodiments, the method promotes phagocytic activity, reduces tumor burden, and improves subject survival.

### BRIEF DESCRIPTION OF THE DRAWINGS

FIGS. 1A-1I show production of human iPSC-derived macrophages. FIG. 1A shows a schematic diagram describing culture conditions required for each differentiation step from human iPSCs to generation of polarized macrophages. Briefly, to produce hematopoietic progenitor cells from human iPSCs, spin EBs are formed after plating ˜8000 human iPSCs per well of a round-bottom 96-well plate in a differentiation medium containing SCF, VEGF and BMP4, as in previous studies from our group. After 6 days of culture, EBs transferred to 6-well plates in media supplemented with M-CSF and IL-3. After about 1-2 weeks, the EBs produce human macrophage progenitor cells (iPSC-MPro). Next, human iPSC-MPro are transferred to new plates in serum-free media containing M-CSF and allow to mature for 5-7 days (iPSC-M0). These iPSC-derived macrophages can then be primed in vitro to either M1 or M2 phenotypes by treating with LPS+IFN-γ or IL-4+IL-13, respectively.

FIG. 1B shows representative images of each differentiation step including modified Giemsa stain of human iPSC-M0 are depicted (scale; 100 μm). FIG. 1C shows a flow cytometric analysis of human iPSCs (TRA-1-81 and SSEA-4) and FIG. 1D shows hematopoietic progenitor cells (EBs; CD34, CD31, CD43 and CD45). FIG. 1E shows a cumulative number of human iPSC-MPro generated in this system demonstrates the ability to continuously produce human iPSC-MPro from undifferentiated human iPSCs for more than 12 weeks at a quantity of ˜1×10⁶ cells/week/well (6-well plate). FIG. 1F shows a phenotypic characterization of human iPSC-MPro over different harvests is shown. FIG. 1G shows a flow cytometric analysis to demonstrate expression of typical antigenic markers CD14, CD11b, CD68, CD86, HLA-DR and SIRP-α on human iPSC-M0 and human PB-M0 indicating that human iPSC-M0 have the same marker phenotype to that of human PB-M0.

FIG. 1H shows RT-qPCR for M1 (CD80, CD40) in human iPSC (M0, M1 and M2). Data represent the mean+/−SEM of n=3 technical replicates. Statistics: one-way ANOVA with multiple comparisons; ***p<0.001. FIG. 1I shows MSD analysis of cytokine expression (TNF-a, IL-6, IL-10, TARC, MDC) in human iPSC-Macs (M0, M1 and M2). Data represent the mean+/−SEM of n=2 biological replicates. Statistics: one-way ANOVA with multiple comparisons; ***p<0.001.

FIG. 2A shows a phagocytosis of human iPSC-derived macrophages toward ovarian cancer cells (A1847). Human iPSC-derived macrophages were co-cultured with ovarian cancer line (A1847) with different E:T ratios in presence of either CD47 antibody (10 μg/ml) or anti-EGFR antibody (μg/ml) or a combination of both antibodies for 2-4 hours. Event of phagocytosis were measured using flow cytometry.

FIGS. 2B-2D show functional assessment of iPSC-Mac. FIG. 2B shows phagocytosis of carboxy late-modified polystyrene-labeled latex beads (2 hours incubation) is used to demonstrate function of iPSC and PB-Macs (M0, M1 and M2) as analyzed by flow cytometry (blue filled: cells treated with 2 μm beads; red filled: untreated cell). FIG. 2C shows representative image of fluorescent microscopy of iPSC-M0 without and with latex beads, respectively is shown (scale: 200 μm). FIG. 2D shows phagocytosis of iPSC-Mac toward ovarian cancer cells (A1847) at different E:T ratios in presence of either anti-CD47 Abs or anti-EGFR Abs or a combination of both for 2-4 hours. Phagocytosis was quantified by flow cytometry. Data represent the mean±SD of n=2 technical replicates. Statistics: two-way ANOVA with Tukey's multiple comparisons; *p<0.05 and ****p<0.0001.

FIGS. 3A-3C show a combination therapy using wild-type human iPSC-macrophages and therapeutic antibodies CD47, and EGFR display superior anti-tumor activity against human ovarian carcinoma model. FIG. 3A shows mice were IP injected with 1×10⁵ luciferase expressing A1847 ovarian cancer cells, 4 days later, received IP injections of 10⁷ human macrophages (1× per week; 3 times), and/or 200 μg anti-CD47 (daily) and/or 1 mg anti-EGFR (2× per week) abs as indicated. Tumor burden was determined by bioluminescent imagining (BLI) at indicated time points after treatment. FIG. 3B shows a tumor burden (total flux) by bioluminescent imaging 10 days after treatment. FIG. 3C shows a Kaplan-Meier analysis of survival of different group of mice therapeutically treated with human iPSC-derived macrophages and therapeutic Abs.

FIGS. 4A-4C show a study on the combination therapy using wild-type human iPSC-macrophages and therapeutic antibodies CD47, and EGFR and its comparison with application of therapeutic antibodies CD47, and EGFR only. FIG. 4A shows mice were IP injected with 1×10⁵ luciferase expressing A1847 ovarian cancer cells, 4 days later, received IP injections of 10⁷ human macrophages (1× per week; 3 times), and/or 200 μg anti-CD47 (daily) and/or 1 mg anti-EGFR (2× per week) Abs as indicated. Tumor burden was determined by bioluminescent imagining (BLI) at indicated time points after treatment. FIG. 4B shows a tumor burden (total flux) by bioluminescent imaging days after treatment. FIG. 4C shows a Kaplan-Meier analysis of survival of different group of mice therapeutically treated with human iPSC-derived macrophages and therapeutic Abs.

FIGS. 5A-5D show application of CAR molecules to the human iPSC-derived macrophages. FIG. 5A shows analysis of phagocytosis of anti-meso-2B4-CD3z iPSC-CARMAs against A1847 ovarian cancer cells. Phagocytic potential of unmodified (WT) human iPSC-macrophages was compared to human iPSC-CARMA expressing signaling domains optimized for NK cell expression (NKG2D-2B4, CD3z). 5×10⁴ of either wild-type human iPSC-macrophages or anti-meso-2B4-CD3z CARMAs were co-cultured with A1847 cells labelled with CellTrace Violet in different E:T ratios for 4 hours and their phagocytic activity was measured by flow cytometry. FIG. 5B show CAR constructs utilized in this study. FIG. 5C shows phagocytic activity of different human iPSC-CARMAs against A1847 ovarian cancer cells. 5×10⁴ anti-meso-iPSC-CARMAs (GFP+) were co-cultured with A1847-cells labelled with CellTrace Violet in different E:T ratios for 4 hours and their phagocytic activity was compared to wild-type (WT, unmodified) iPSC-macrophages. These studies demonstrate markedly improved phagocytic activity by the iPSC-CARMAs expressing the anti-meso-Bai1 CAR construct. FIG. 5D shows a combination of anti-CD47 antibody and candidate CARMA (Bai1-CARMA) improves phagocytosis of ovarian tumor cells. Wild-type macrophages were utilized as control.

FIGS. 6A-6C show functional assessment of CAR expressing iPSC-Mac. FIG. 6A shows a schematic representation of the transposon vector encoding macrophage specific CARs. Transmembrane (TM): CD8a, and stimulation domain (SD): CD3ζ or Bai1. iPSCs were transfected with CARs subcloned into a Piggy Bac transposon vector, then differentiated into iPSC-CarMacs using our differentiation protocol. FIG. 6B shows flow cytometric analysis demonstrates stable GFP expression with no CD32-CAR expression and low Bai1-CAR expression in iPSC-Macs. FIG. 6C shows phagocytosis of un-modified and modified iPSC-Mac toward ovarian cancer cells (A1847) at different E:T ratios for 2-4 hours. Phagocytosis was quantified by flow cytometry. Data represent the mean+/−SEM of n=2 technical replicates. Statistics: two-way ANOVA with Tukey's multiple comparisons; ns P>0.05, ***p<0.001, and ****p<0.0001.

FIGS. 7A-7C show combination therapy using Bai1-iPSC-CarMac and therapeutic antibody CD47, display superior anti-tumor activity against human ovarian carcinoma model. FIG. 7A shows NSG-SGM3 mice were injected IP with 1×10⁵ luciferase expressing A1847 ovarian cancer cells, 4 days later, received IP injections of 10⁷ human macrophages (1× per week for 3 weeks), and 200 μg anti-CD47 (daily). Tumor burden was determined by bioluminescent imagining (BLI). FIG. 7B shows tumor burden (total flux) by BLI 23 days after treatment. Statistics: two-tail t test; **p<0.01. FIG. 7C shows a Kaplan-Meier curve representing the percent survival of the experimental groups. Statistics: log-rank (Mantel-Cox) test; **p<0.01.

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The invention provides human iPSC-derived macrophages, and methods for the manufacture thereof, as described herein. In embodiments, the human iPSC-derived macrophage further comprises a chimeric antigenic receptor (CAR) expressed thereon. In embodiments, the CAR is Bai1, MegF10 or MerTK. In embodiments, the invention promotes phagocytosis of cancer cells, such as ovarian cancer cells.

The invention provides a method of treatment comprising administering to a subject in need thereof an effective amount of a pharmaceutically acceptable composition comprising human iPSC-derived macrophage cells. In embodiments, the human iPSC-derived macrophages comprise a chimeric antigenic receptor (CAR) expressed thereon. In embodiments, the CAR is Bai1, MegF10 or MerTK.

In embodiments, the treatment is for a cancer. In embodiments, the cancer is ovarian. In embodiments, the treatment is for fibrosis, autoimmune disorders, or senescent cells.

In embodiments, the method of treatment further comprises administering to the subject an effective amount of an antibody specific for the cancer. In embodiments, the antibody is an anti-CD47 or an anti-EGFR antibody.

In embodiments, the method promotes macrophage phagocytic activity. In embodiments, the method reduces tumor burden.

In embodiments, the method of treatment further comprises administering to the subject an effective amount of an immune-stimulating agent, a TLR agonist, or a checkpoint inhibitor.

In embodiments, the method of treatment further comprises administering to the subject an effective amount of CAR-T cells, NK cells or CAR-NK cells.

In embodiments, the invention provides a pharmaceutically acceptable composition comprising human iPSC-derived macrophages. In embodiments, the human iPSC-derived macrophages comprise a chimeric antigenic receptor (CAR) expressed thereon. In embodiments, the CAR is Bai1, MegF10 or MerTK.

In embodiments, the invention provides methods for the manufacture of human iPSC-derived macrophages comprising, producing hematopoietic progenitor cells from human iPSCs, by spinning EBs formed after plating human iPSCs in a differentiation medium containing the stem cell factor (SCF), vascular endothelial growth factor (VEGF) and bone morphogenic protein 4 (BMP4). After about 6 days of culture, the method further comprises transferring EBs to well plates in media supplemented with M-CSF and IL-3. After about 1-2 weeks, the EBs produce human macrophage progenitor cells (iPSC-MPro). Next, human iPSC-MPro are transferred to new plates in serum-free media containing M-CSF and allowed to mature for 5-7 days (iPSC-M0). The human iPSC-MPro have a phenotypic expression of typical antigenic markers CD14, CD11b, CD68, CD86, HLA-DR and SIRP-α on human iPSC-M0 and human PB-M0. These iPSC-derived macrophages can then be primed in vitro to either M1 or M2 phenotypes by treating with LPS+IFN-g or IL-4+IL-13, respectively. Production of iPSC-derived macrophage cells can be done under cGMP conditions at clinical scale.

The invention may be applied to therapies using the CAR-macrophage system include, but are not limited to, targets for solid tumors and hematological malignancies. Examples of targets for solid tumors include, but are not limited to: Bai1, MegF10, MerTK, AChR (Fetal acetylcholine receptor), B7-H4, CAIX (carbonic anhydrase IX), CD133 (prominin-1), CD44v6, CD47 (integrin associated protein or IAP), CD70) (used in multiple disease categories), CEA (carcinoembryonic antigen), c-Met (c-mesenchymal-epithelial transition factor), DLL3 (Delta-like 3), EGFR (epidermal growth factor receptor), EGFRVIII (type III variant epidermal growth factor receptor, EpCAM (epithelial cell adhesion molecule), EphA2 (Erythropoetin producing hepatocellular carcinoma A2), ErbB2, FAP (fibroblast activation protein), FRa (folate receptor alpha), Frizzled 7 (Fzd7), GD2 (Ganglioside GD2), GPC3 (Glypican-3), GUCY2C (Guanylyl cyclase C), HER1 (human epidermal growth factor receptor 1), HER2 (ErbB2, human epidermal growth factor 2), ICAM-1 (intercellular adhesion molecule 1), IL11Rα (interleukin 11 receptor a), IL13Rα2 (interleukin 13 receptor a2), L1-CAM (human L1 cell adhesion molecule, CD171), LeY (Lewis Y antigen), MAGE (Melanoma-associated antigen), MCAM (CD146) (melanoma cell adhesion molecule), Mesothelin, MUC1 (mucin 1), MUC16 ecto (mucin 16), NKG2DLs (natural killer group 2 member D ligands), NY-ESO-1 (Cancer/testis antigen 1), PD-L1 (B7-H1) (CD274), PSCA (prostate stem cells antigen), PSMA (prostate-specific membrane antigen), RORI (receptor tyrosine kinase-like orphan receptor) (used in multiple disease categories), TAG72 (tumor-associated glycoproteins-72), and VEGFR (Vascular endothelial growth factor receptor 1).

The practice of the present invention may employ conventional techniques of molecular biology (including recombinant techniques), microbiology, cell biology, biochemistry and immunology, which are within the skill of the art. Such techniques are explained fully in the literature, such as Molecular Cloning: A Laboratory Manual, second edition (Sambrook et al, 1989) Cold Spring Harbor Press; Oligonucleotide Synthesis (M J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C C. Blackwell, eds.); Gene Transfer Vectors for Mammalian Cells (J. M. Miller and M. P. Calos, eds., 1987); Current Protocols in Molecular Biology (F. M. Ausubel et al, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al, eds., J.B. Lippincott Company, 1993). Although any methods and materials similar or equivalent to those described herein can be used in the practice of the present invention, the exemplary methods, devices, and materials are described herein. For the purposes of the present disclosure, the following terms are defined below. Additional definitions are set forth throughout this disclosure.

As used herein, the terms “comprises,” “comprising,” “includes,” “including.” “has,” “having.” “contains”, “containing,” “characterized by,” or any other variation thereof, are intended to encompass a non-exclusive inclusion, subject to any limitation explicitly indicated otherwise, of the recited components. For example, a protein, a pharmaceutical composition, and/or a method that “comprises” a list of elements (e.g., components, features, or steps) is not necessarily limited to only those elements (or components or steps), but may include other elements (or components or steps) not expressly listed or inherent to the protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein, and unless otherwise specified, the term “subject” or “patient” is defined herein to include animals such as mammals, including, but not limited to, primates (e.g., humans), cows, sheep, goats, horses, dogs, cats, rabbits, rats, mice, and the like. In specific embodiments, the subject is a human. The terms “subject” and “patient” are used interchangeably herein in reference, for example, to a mammalian subject, such as a human.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soy bean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

As used herein, “induced pluripotent stem cell” or “iPSC cell” or “iPSCs” are used to refer to cells, derived from somatic cells, that have been reprogrammed back to an pluripotent state that are capable of proliferation, selectable differentiation, and maturation.

As used herein, a composition containing a “purified cell population” or “purified cell composition” means that at least 30%, 50%, 60%, typically at least 70%, and more preferably 80%, 90%, 95%, 98%, 99%, or more of the cells in the composition are of the identified type.

Generally, techniques for differentiating an induced pluripotent cell involve modulation of specific cellular pathways, either directly or indirectly, using polynucleotide-, polypeptide- and/or small molecule-based approaches. The developmental potency of a cell may be modulated, for example, by contacting a cell with one or more modulators. “Contacting”, as used herein, can involve culturing cells in the presence of one or more factors (such as, for example, small molecules, proteins, peptides, etc.). In some embodiments, a cell is contacted with one or more agents to induce cell differentiation. Such contact, may occur for example, by introducing the one or more agents to the cell during in vitro culture. Thus, contact may occur by introducing the one or more agents to the cell in a nutrient cell culture medium. The cell may be maintained in the culture medium comprising one or more agents for a period sufficient for the cell to achieve the differentiation phenotype that is desired.

Differentiation of stem cells requires a change in the culture system, such as changing the stimuli agents in the culture medium or the physical state of the cells. A conventional strategy utilizes the formation of embryoid bodies (EBs) as a common and critical intermediate to initiate the lineage-specific differentiation. EBs are three-dimensional clusters that have been shown to mimic embryo development as they give rise to numerous lineages within their three-dimensional area. Through the differentiation process simple EBs (for example, aggregated pluripotent stem cells elicited to differentiate) continue maturation and develop into a cystic EB at which time, they are further processed to continue differentiation. EB formation is initiated by bringing pluripotent stem cells into close proximity with one another in three-dimensional multilayered clusters of cells. Typically, this is achieved by one of several methods including allowing pluripotent cells to sediment in liquid droplets, sedimenting cells into “U” bottomed well-plates or by mechanical agitation. To promote EB development, the pluripotent stem cell aggregates require further differentiation cues, as aggregates maintained in pluripotent culture maintenance medium do not form proper EBs. This may be followed by additional stimulation differentiating the iPSCs.

Illustrative methods for making and using engineered cells are provided in Int'l Pat. Appl. Nos. WO 2013/163171 A1, WO 2017/078807 A1, and WO 2018/147801 the disclosures of which are incorporated by reference herein in their entireties.

As used herein, “differentiate” or “differentiated” are used to refer to the process and conditions by which immature (unspecialized) cells acquire characteristics becoming mature (specialized) cells thereby acquiring particular form and function. Stem cells (unspecialized) are often exposed to varying conditions (e.g., growth factors and morphogenic factors) to induce specified lineage commitment, or differentiation, of said stem cells. The process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

In some embodiments, one or more of the media of the culture platform is a feeder-free environment, and optionally is substantially free of cytokines and/or growth factors. In some embodiments, the cell culture media contains supplements such as serums, extracts, growth factors, hormones, cytokines and the like. Generally, the culture platform comprises one or more of stage specific feeder-free, serum-free media, each of which further comprises one or more of the followings: nutrients/extracts, growth factors, hormones, cytokines and medium additives. Suitable nutrients/extracts may include, for example, DMEM/F-12 (Dulbecco's Modified Eagle Medium/Nutrient Mixture F-12), which is a widely used basal medium for supporting the growth of many different mammalian cells; KOSR (knockout serum replacement); L-glut; NEAA (Non-Essential Amino Acids). Other medium additives may include, but not limited to, MTG, ITS, (ME, anti-oxidants (for example, ascorbic acid). In some embodiments, a culture medium of the present invention comprises one or more of the following cytokines or growth factors: epidermal growth factor (EGF), acidic fibroblast growth factor (aFGF), basic fibroblast growth factor (bFGF), leukemia inhibitory factor (LIF), hepatocyte growth factor (HGF), insulin-like growth factor 1 (IGF-1), insulin-like growth factor 2 (IGF-2), keratinocyte growth factor (KGF), nerve growth factor (NGF), platelet-derived growth factor (PDGF), transforming growth factor beta (TGF-β), bone morphogenetic protein (BMP4), vascular endothelial cell growth factor (VEGF) transferrin, various interleukins (such as IL-1 through IL-18), various colony-stimulating factors (such as granulocyte/macrophage colony-stimulating factor (GM-CSF)), various interferons (such as IFN-γ) and other cytokines having effects upon stem cells such as stem cell factor (SCF) and erythropoietin (EPO). These cytokines may be obtained commercially, for example from R&D Systems (Minneapolis, Minn.), and may be either natural or recombinant. In some other embodiments, the culture medium of the present invention comprises one or more of bone morphogenetic protein (BMP4), insulin-like growth factor-1 (IGF-1), basic fibroblast growth factor (bFGF), vascular endothelial growth factor (VEGF), hematopoietic growth factor (for example, SCF, GMCSF, GCSF, EPO, IL3, TPO, EPO), Fms-Related Tyrosine Kinase 3 Ligand (Flt3L); and one or more cytokines from Leukemia inhibitory factor (LIF), IL3, IL6, IL7, IL11, IL15. In some embodiments, the growth factors/mitogens and cytokines are stage and/or cell type specific in concentrations that are determined empirically or as guided by the established cytokine art.

“Culture” or “cell culture” refers to the maintenance, growth and/or differentiation of cells in an in vitro environment. “Cell culture media,” “culture media” (singular “medium” in each case), “supplement” and “media supplement” refer to nutritive compositions that cultivate cell cultures.

“Cultivate,” or “maintain,” refers to the sustaining, propagating (growing) and/or differentiating of cells outside of tissue or the body, for example in a sterile plastic (or coated plastic) cell culture dish or flask. “Cultivation,” or “maintaining,” may utilize a culture medium as a source of nutrients, hormones and/or other factors helpful to propagate and/or sustain the cells.

Multipotent hematopoietic stem cells provide the basis of two major progenitor cell lineages. The first cell lineage is the common lymphoid progenitor cell lineage, wherein a multipotent hematopoietic stem cell (hemocytoblast) differentiates into a lymphoid progenitor cell, which has the capability to further differentiate into a natural killer cell, T lymphocyte, or B lymphocyte; or differentiate even further from a B lymphocyte to a plasma cell. The other major cell lineage is the common myeloid progenitor cell lineage, wherein a hemocytoblast differentiates into a myeloid progenitor cell, which has the capability to further differentiate into a megakaryocyte, erythrocyte, platelet, mast cell, or myeloblast; or differentiate even further from a myeloblast to a basophil, neutrophil, eosinophil, or monocyte; or yet further differentiate from a monocyte to a macrophage.

In some embodiments, the treatment is for a malignancy that may include Acute Lymphoblastic Leukemia (ALL), Acute Myeloid Leukemia (AML), Adrenocortical Carcinoma, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Atypical Teratoid/Rhabdoid Tumor, Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors, Burkitt Lymphoma, Carcinoid Tumor, Carcinoma, Cardiac Tumors, Atypical Teratoid/Rhabdoid Tumor, Medulloblastoma, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Endometrial Cancer, Ependymoma, Esophageal Cancer, Esthesioneuroblastoma, Ewing Sarcoma, Extracranial Germ Cell Tumor, Extragonadal Germ Cell Tumor, Intraocular Melanoma, Retinoblastoma, Fallopian Tube Cancer, Fibrous Histiocytoma of Bone, Osteosarcoma, Gallbladder Cancer, Gastric Cancer, Gastrointestinal Carcinoid Tumor, Gastrointestinal Stromal Tumors (GIST) (Soft Tissue Sarcoma), Germ Cell Tumors, Central Nervous System Germ Cell Tumors, Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Hepatocellular Cancer, Histiocytosis (Langerhans Cell), Hodgkin Lymphoma. Hypopharyngeal Cancer, Islet Cell Tumors, Pancreatic Neuroendocrine Tumors, Kaposi Sarcoma (Soft Tissue Sarcoma), Renal Cell Cancer, Langerhans Cell Histiocytosis, Laryngeal Cancer, Leukemia, Lip and Oral Cavity Cancer, Liver Cancer, Lung Cancer (Non-Small Cell, Small Cell, Pleuropulmonary Blastoma, and Tracheobronchial Tumor), Lymphoma, Male Breast Cancer, Malignant Fibrous Histiocytoma of Bone and Osteosarcoma, Merkel Cell Carcinoma, Mesothelioma, Metastatic Cancer, Metastatic Squamous Neck Cancer with Occult Primary, Midline Tract Carcinoma With NUT Gene Changes, Oropharyngeal Cancer, Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Chronic Myelogenous Leukemia (CML), Myeloid Leukemia, Acute (AML), Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System (CNS) Lymphoma, Primary Peritoneal Cancer, Prostate Cancer, Recurrent Cancer, Rhabdomyosarcoma, Salivary Gland Cancer, Vascular Tumors, Small Intestine Cancer, Soft Tissue Sarcoma, T-Cell Lymphoma, Thymoma and Thymic Carcinoma, Transitional Cell Cancer of the Renal Pelvis and Ureter, Vaginal Cancer, Vulvar Cancer, or Wilms Tumor.

In some embodiments, the condition for treatment may comprise tumor-associated antigens. In some embodiments, the malignancy may comprise a cell marker characteristic of a malignancy. In some embodiments, the cell marker characteristic of a malignancy is a tumor-associated antigen, receptor, or other protein or structure attributed to cells with cancerous phenotypes.

Illustrative tumor-associated antigens for CAR targetting include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGEA1, MAGE-A2, MAGE-A3, MAGE-A4, MAGE-A6, MAGE-A10, MAGE-A12, MART-1, BAGE, DAM-6, DAM-10, GAGE-1, GAGE-2, GAGE-8, GAGE-3, GAGE-4, GAGE-5, GAGE-6, GAGE-7B, NA88-A, MART-1, MC1R, GplOO, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, β-catenin/m, Caspase-8/m, CEA, CDK-4/m, ELF2M, GnT-V. G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (e.g., such as EGFRVIII), platelet derived growth factor receptor (PDGFR), vascular endothelial growth factor receptor (VEGFR), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notchl-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AMLI, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLACI, GM3, BORIS, Tn, GLoboH, NY-BR-1, RGsS, SART3, STn, PAX5, OY-TES1, sperm protein 17, LCK, HMWMAA, AKAP-4, SSX2, XAGE 1, B7H3, legumain, TIE2, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1, melanocyte melanoma lineage antigens (e.g., MART-1/Melan-A, gp75, mda-7, tyrosinase and tyrosinase-related protein), HER-2/neu, and idiotypes.

In some embodiments, the malignancy, or cells thereto, exhibit CD19, CD20, Her2, CD19, CD319/CS1, RORI, CD20, CD5, CD7, CD22, CD70, CD30, BCMA, CD25, NKG2D ligands, MICA/MICB, carcinoembryonic antigen, alphafetoprotein, CA-125, MUC-1, epithelial tumor antigen, melanoma-associated antigen, mutated p53, mutated ras, HER2/Neu, ERBB2, folate binding protein, HIV-1 envelope glycoprotein gpl20, HIV-1 envelope glycoprotein gpl41, GD2, CD123, CD33, CD30, CD56, c-Met, mesothelin, GD3, HERV-K, IL-IlRalpha, kappa chain, lambda chain, CSPG4, ERBB2, WT-1, EGFRVIII, TRAIL/DR4, VEGFR2, PTK-7, B7H3, PD-L1, CD38, CLL-1, LeY, CAIX, CD133, CD171, GPC3, CEA, Ep-CAM, EphA2, FAP, HPV16-E6, IL13Ra2, MAGEA3, MAGEA4, MARTI, MUC16, NY-ESO-1 and/or PSCA, CLL-1/CLEC12A, BCMA, TROP2, Nectin-4, CD79b, CD2, CD3, CD4, PD-1, KIR2DL3, ALPPL2, or CSP1.

The term “antibody” as used herein encompasses monoclonal antibodies (including full length monoclonal antibodies), polyclonal antibodies, multi-specific antibodies (e.g., bi-specific antibodies), and antibody fragments so long as they exhibit the desired biological activity of binding to a target antigenic site and its isoforms of interest. The term “antibody fragments” comprise a portion of a full length antibody, generally the antigen binding or variable region thereof. The term “antibody” as used herein encompasses any antibodies derived from any species and resources, including but not limited to, human antibody, rat antibody, mouse antibody, rabbit antibody, and so on, and can be synthetically made or naturally-occurring.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The “monoclonal antibodies” may also be isolated from phage antibody libraries using the techniques known in the art.

The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. As used herein, a “chimeric protein” or “fusion protein” comprises a first polypeptide operatively linked to a second polypeptide. Chimeric proteins may optionally comprise a third, fourth or fifth or other polypeptide operatively linked to a first or second polypeptide. Chimeric proteins may comprise two or more different polypeptides. Chimeric proteins may comprise multiple copies of the same polypeptide. Chimeric proteins may also comprise one or more mutations in one or more of the polypeptides. Methods for making chimeric proteins are well known in the art.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-polyacrylamide gel electrophoresis under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

### Examples

The goal of the present example was to investigate whether human iPSC-derived macrophages can be therapeutically utilized in ovarian cancer mouse model when combined with therapeutic antibodies and whether addition of CAR molecule with different signaling domains would improve anti-tumor activity in vitro and in vivo.

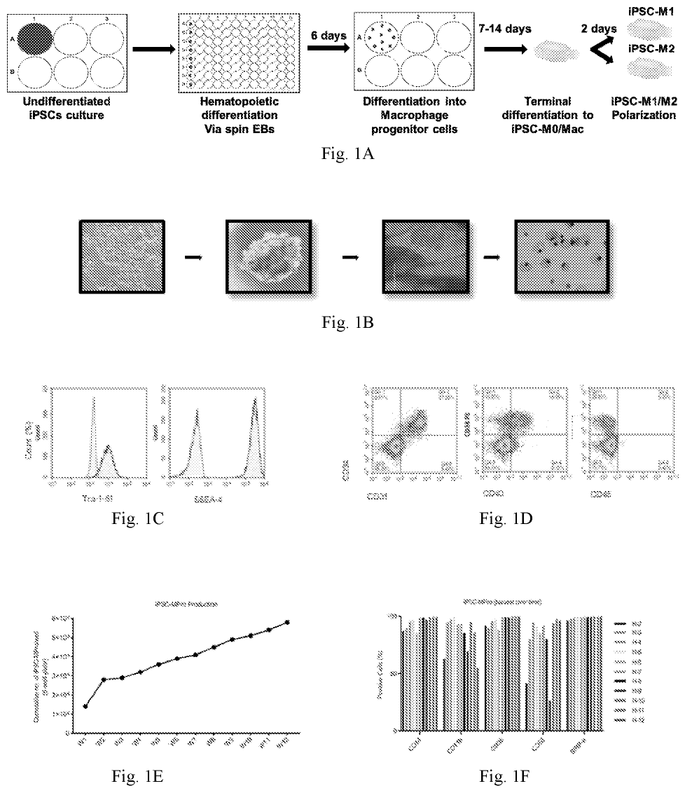

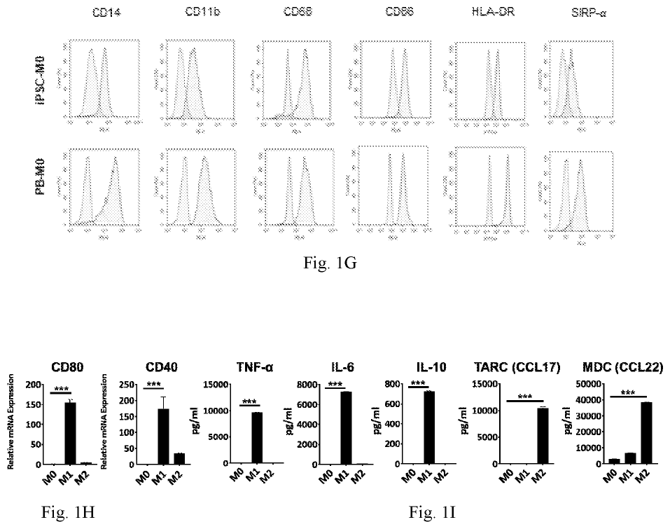

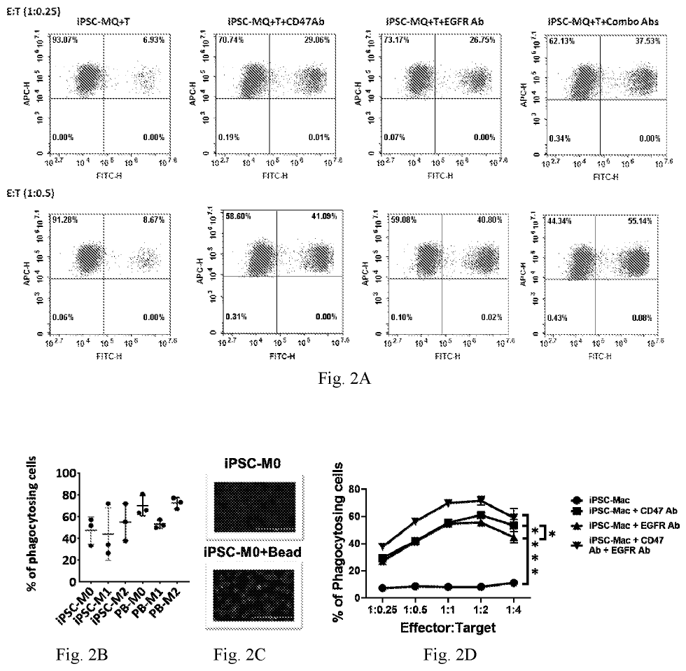

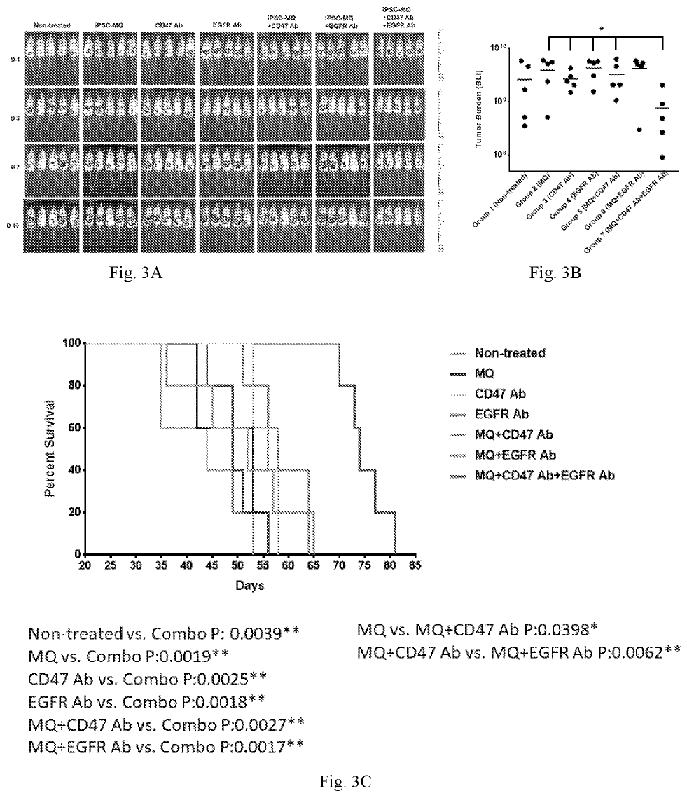

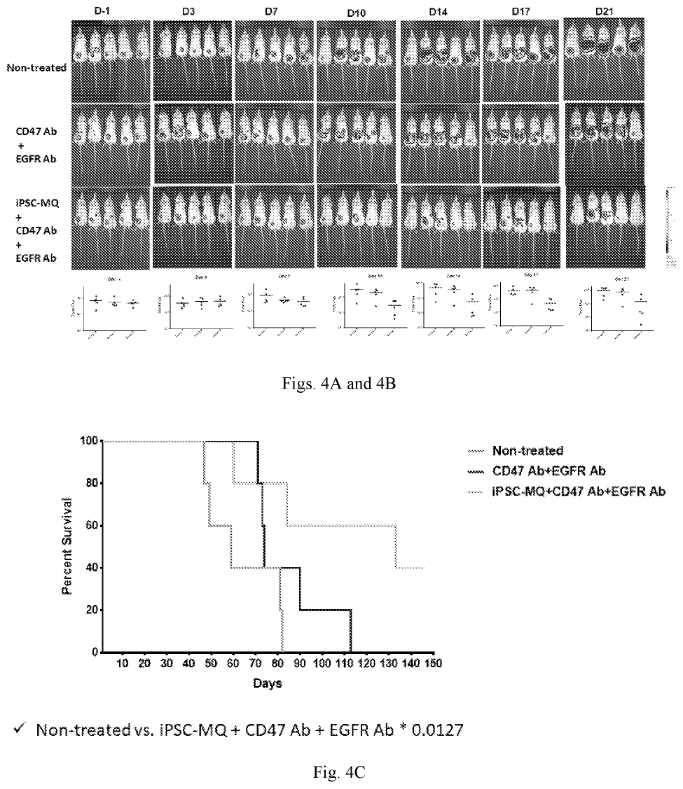

Human iPSCs serve as a ready source of monocytes and macrophages and can be easily scaled up for therapeutic applications (FIG. 1A). The data demonstrated that administration of human iPSC-derived macrophages with therapeutic antibodies (for example: anti-CD47 and anti-EGFR antibodies) improved phagocytic activity in vitro, reduced tumor burden in vivo and improved survival with an ovarian cancer xenograft model.

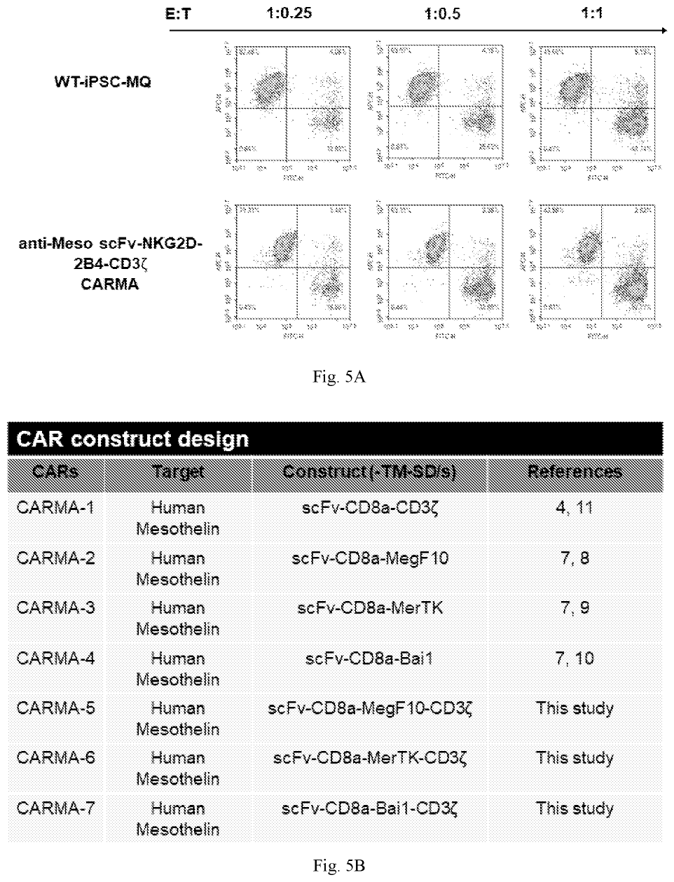

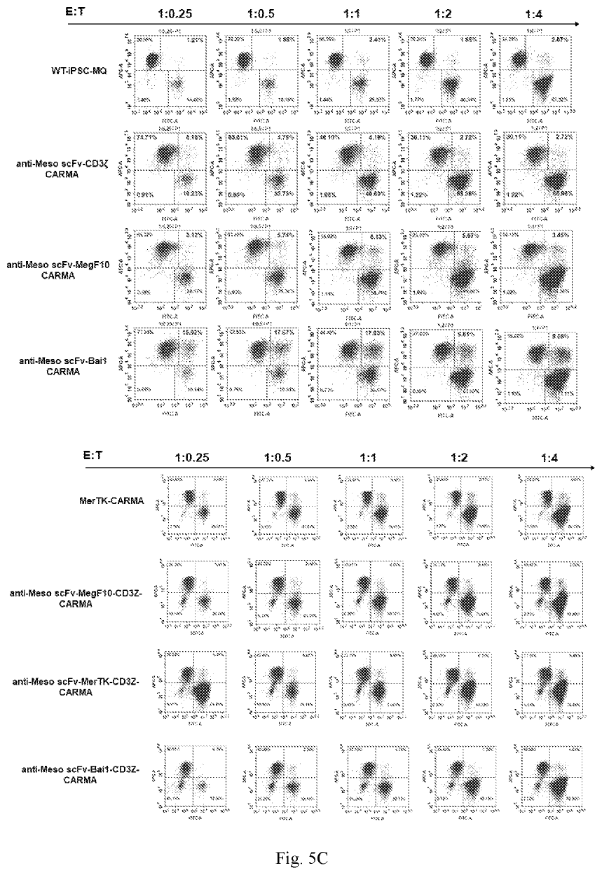

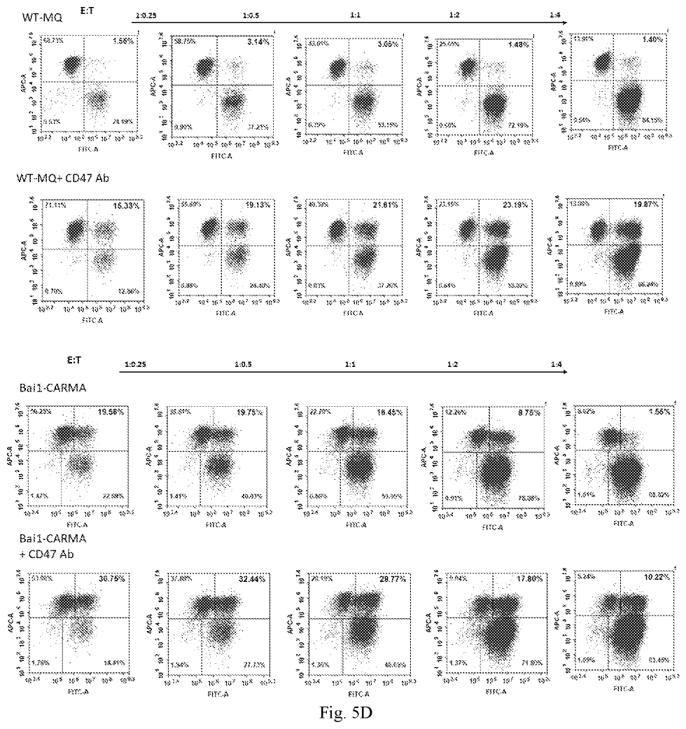

Second, the invention provides macrophage-specific CARs via testing distinct intracellular signaling domains known to regulate macrophage activity to optimize signaling components of cell specific CARs to promote improved anti-tumor activity (5). As noted, these with engineered iPSC-derived CAR-NK cells that have now been translated into clinical trials (NCT04245722). These CARs had been specifically designed for NK cells and not optimized for macrophage cell signaling as differentiating those human iPSCs expressing anti-meso-2B4-CD3z CARs to human iPSC-derived macrophages still did not conferred improved phagocytic capacity (FIG. 5A). Therefore, optimization of signaling domains for a iPSC-CAR-macrophage system was still needed.

Studies in this project utilized human iPSCs to generate human iPSC-CAR macrophages (human iPSC-CARMAs). Human iPSCs were genetically engineered to express CARs against mesothelin tumor antigen. Of note, other gene transfer methods (viral or transposon based) can be used routinely to genetically modify human iPSCs. Here, macrophage-optimized CARs were identified to produce improved iPSC-derived CAR-expressing macrophages (iPSC-CARMAs). To identify signaling domains capable of promoting phagocytosis, the assay screened a panel of phagocytic receptors including CD3z, MegF10, MerTK and Bai1 (FIG. 5B) (6, 7) and their respective human iPSC-CARMAs were evaluated in vitro for their phagocytic capability (FIG. 5C,D). MegF10 (Multiple epidermal growth factor-like domains protein 10) signals via GULP and Syk to elicit Rac1 activation (8), MerTK (Tyrosine-protein kinase Mer) may associate with integrins for signaling through FAK phosphorylation (9), Bai 1 (Adhesion G protein-coupled receptor B1; ADGRB1) interacts through ELMO-Dock180-Rac1 module to mediate actin cytoskeletal reorganization during engulfment (10).

The data demonstrated that application Bali-CAR molecule (human iPSCBai1-CARMAs) was best able to promote macrophage phagocytosis of ovarian cancer cells in vitro. Other novel CAR constructs including MegF10 and MerTK containing CARs also demonstrated some benefit.

Together, these studies provide support for this high impact approach to produce a new, targeted off the-shelf immune therapy that can be utilized to treat refractory solid tumors.

Human iPSCs serve as a ready source of monocytes and macrophages and can be easily scaled up for therapeutic applications (FIG. 1). Our data demonstrate that administration of human iPSC-derived macrophages with therapeutic antibodies (CD47 and EGFR antibodies) improved phagocytic activity in vitro and reduced tumor burden in vivo using ovarian cancer xenograft model.

This study demonstrates that application of macrophage-specific CAR molecule, promote macrophage phagocytosis of ovarian cancer cells in vitro. This approach can be used to target any tumor antigen, as has now become standard with other immunotherapy products (e.g., CAR-T cells and CAR-NK cells). This approach can be used for iPSC-derived macrophages (with or without CAR expression) to also treat non-malignant diseases (fibrosis, autoimmune disorders, senescent cells, for example).

This study is also the first to utilize human iPSC-derived macrophages in combination with therapeutic antibodies (e.g., CD47 and EGFR antibodies) to demonstrate improved anti-tumor activity in vitro and in vivo. This is also the first work to use macrophage-specific CARs to improve activity of iPSC-derived macrophages.

Recent studies by Klichinsky et al have expressed CARs into peripheral blood-derived macrophages for treatment of breast and ovarian cancers in xenograft mouse models (4). In those studies, human macrophages were engineered with an adenoviral vector to express an anti-HER-2 CAR with the CD3z intracellular domain. CD3z has homology to the Fc common γ-chain, FcεRI-γ, a canonical signaling molecule for antibody-dependent cellular phagocytosis (ADCP) in macrophages. They demonstrated that single infusion of 7×10⁶ human CAR-expressing macrophages decreased tumor burden and prolonged overall survival in two solid tumor xenograft mouse model. This product recently entered in phase I clinical trial in patients with recurrent or metastatic HER-2 overexpressing solid tumors (NCT04660929). However, the complexity associated with manufacturing the CAR macrophage product from peripheral blood to obtain enough cells (10⁹-10¹⁰) and to efficiently engineering them with the CAR construct demonstrates the need for an alternative approach for development of CAR macrophage-based therapies for clinical application.

Additionally, alternatives to a CAR with only the CD3z chain as the intracellular signaling component, can be used. Zhang et al. demonstrated that introduction of anti-CD19 or anti-meso CARs containing the CD3z signaling domain into human iPSCs using lentivirus transfection resulted in human iPSC-derived macrophages with increased phagocytosis toward respective tumor cells as well as effective anti-tumor activity in vivo against both liquid and solid tumors (11). This work did not use macrophage-optimized CARs as was done here.

The present study investigated different signaling domains involved in macrophage function (CD3z, MegF10, MerTK, Bai1) (FIG. 5B) to be present in CAR expressing macrophages (CARMAs). Piggy Bac transposon vector was utilized as a non-viral delivery platform to stably transfect human iPSCs, though other gene transfer methods can also be used. Human iPSC-CARMAs expressing Bai1 signaling domain showed the best phagocytic activity toward solid tumor (ovarian cancer cells) (FIG. 5C-D).

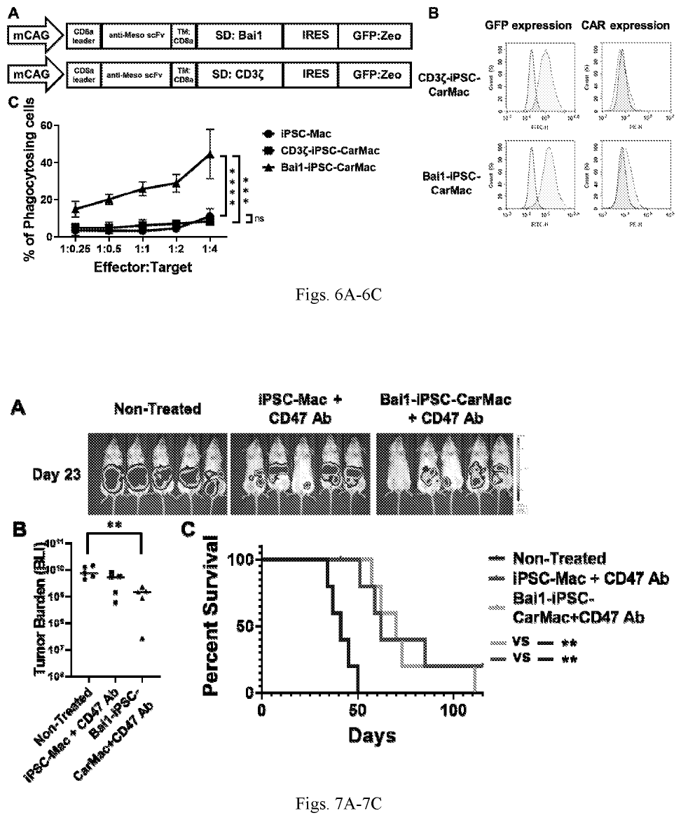

FIGS. 6A-6C show functional assessment of CAR expressing iPSC-Mac. FIG. 6A shows a schematic representation of the transposon vector encoding macrophage specific CARs. Transmembrane (TM): CD8a, and stimulation domain (SD): CD3ζ or Bai1. iPSCs were transfected with CARs subcloned into a Piggy Bac transposon vector, then differentiated into iPSC-CarMacs using our differentiation protocol. FIG. 6B shows flow cytometric analysis demonstrates stable GFP expression with no CD3ζ-CAR expression and low Bai1-CAR expression in iPSC-Macs. FIG. 6C shows phagocytosis of un-modified and modified iPSC-Mac toward ovarian cancer cells (A1847) at different E:T ratios for 2-4 hours. Phagocytosis was quantified by flow cytometry. Data represent the mean+/−SEM of n=2 technical replicates. Statistics: two-way ANOVA with Tukey's multiple comparisons; ns P>0.05, ***p<0.001, and ****p<0.0001.

FIGS. 7A-7C show combination therapy using Bai1-iPSC-CarMac and therapeutic antibody CD47, display superior anti-tumor activity against human ovarian carcinoma model. FIG. 7A shows NSG-SGM3 mice were injected IP with 1×10⁵ luciferase expressing A1847 ovarian cancer cells, 4 days later, received IP injections of 10⁷ human macrophages (1× per week for 3 weeks), and 200 μg anti-CD47 (daily). Tumor burden was determined by bioluminescent imagining (BLI). FIG. 7B shows tumor burden (total flux) by BLI 23 days after treatment. Statistics: two-tail t test; **p<0.01. FIG. 7C shows a Kaplan-Meier curve representing the percent survival of the experimental groups. Statistics: log-rank (Mantel-Cox) test; **p<0.01.

Human iPSC-derived macrophages express different FC receptors (FcRs) necessary to mediate phagocytosis of antigens opsonized with therapeutic antibodies. Administration of therapeutic antibodies (e.g., EGFR antibody here) may cross-link the FcRs more efficiently and trigger signals which lead to antibody-mediated phagocytosis of tumor cells.

Tumor cells express signal (CD47) which upon interaction with its partner (SIRP-a) on the surface of macrophage, prevent phagocytosis. Administration of CD47 antibody in combination with therapeutic antibodies, induces more potent phagocytosis of tumor cells by macrophages. Other therapeutic antibodies can be used combined with the iPSC-macrophages.

Moreover, application of chimeric antigenic receptors (CARs) on the surface of macrophages with optimal signaling domain for activity in macrophages, lead to targeted delivery of macrophages and improved phagocytosis toward cancer cells.

Taken together, the data supports application of human iPSC-derived macrophages as a novel, off-the-shelf cell-based therapeutic for treatment of solid and potentially hematologic malignancies. This regimen can be applied either as stand-alone approach when CAR-Macrophages are used, or as combination therapy with therapeutic antibodies.

IPSC-derived macrophages can be utilized for improved cancer therapies. These cells can be combined with antibodies (anti-CD47, tumor antigen targets, and others) or other immune-stimulating agents such as TLR agonists, checkpoint inhibitors, for example. These iPSC-macrophages can be administered as the only cell type, or combined with other cell-based therapies such as CAR-T cells, NK cells or CAR-NK cells. CAR-expressing iPSC-derived macrophages can also be utilized for improved cancer therapy. These iPSC-CAR macrophages can be administered by themselves, combined with antibodies or other immune stimulating agents or other anti-cancer cell products, as above. These iPSC-derived macrophages (with or without CAR expression) can also be used to target and eliminate pathogenic cells in non-malignant diseases such as fibrotic disorders, autoimmune diseases, or senescent cells (i.e. aging).

These and other specific embodiments of the invention will be apparent to those skilled in the art upon a review of the present specification and non-limiting examples described herein.

### REFERENCES

1. Biswas S K, Allavena P, Mantovani A. Tumor-associated macrophages: functional diversity, clinical significance, and open questions. Seminars in immunopathology. 2013; 35(5):585-600.
2. Noy R, Pollard J W. Tumor-associated macrophages: from mechanisms to therapy. Immunity. 2014; 41(1):49-61.
3. Weiskopf K, Weissman I L. Macrophages are critical effectors of antibody therapies for cancer. mAbs. 2015; 7(2): 303-10.
4. Klichinsky M, Ruella M, Shestova O, Lu X M, Best A, Zeeman M, et al. Human chimeric antigen receptor macrophages for cancer immunotherapy. Nature biotechnology. 2020; 38(8):947-53.
5. Li Y, Hermanson D L, Moriarity B S, Kaufman D S. Human iPSC-Derived N K Cells Engineered with Chimeric Antigen Receptors Enhance Anti-tumor Activity. Cell stem cell. 2018; 23(2):181-92.e5.
6. Penberthy K K, Ravichandran K S. Apoptotic cell recognition receptors and scavenger receptors. Immunological reviews. 2016; 269(1):44-59.
7. Morrissey M A, Williamson A P, Steinbach A M, Roberts E W, Kern N, Headley M B, et al. Chimeric antigen receptors that trigger phagocytosis. eLife. 2018; 7.
8. Singh T D, Park S Y, Bae J S, Yun Y, Bae Y C, Park R W, et al. MEGF10 functions as a receptor for the uptake of amyloid-β. FEBS letters. 2010; 584(18):3936-42.
9. Wu Y, Singh S, Georgescu M M, Birge R B. A role for Mer tyrosine kinase in alphavbeta5 integrin mediated phagocytosis of apoptotic cells. Journal of cell science. 2005; 118(Pt 3):539-53.
10. Das S, Owen K A, Ly K T, Park D, Black S G, Wilson J M, et al. Brain angiogenesis inhibitor 1 (BAI1) is a pattern recognition receptor that mediates macrophage binding and engulfment of Gram-negative bacteria. PNAS USA. 2011; 108(5):2136-41.
11. Zhang L, Tian L, Dai X, Yu H, Wang J, Lei A, et al. Induced Pluripotent Stem Cell-derived CARMacrophage Cells with Antigen-dependent Anti-Cancer Cell Functions for Liquid and Solid Tumors. BioRxiv 2020.03.28.011270.

What is claimed is:

1. A method of treatment comprising administering to a subject in need thereof an effective amount of a pharmaceutically acceptable composition comprising human iPSC-derived macrophage cells.

2. The method of claim 1, wherein the human iPSC-derived macrophages comprise a chimeric antigenic receptor (CAR) expressed thereon.

3. The method of claim 2, wherein the CAR is Bai1, MegF10 or MerTK.

4. The method of claim 1, wherein the treatment is for a cancer.

5. The method of claim 1, wherein the cancer is ovarian.

6. The method of claim 1, wherein the treatment is for fibrosis, autoimmune disorders, or senescent cells.

7. The method of claim 4, wherein the method further comprises administering to the subject an effective amount of an antibody specific for the cancer.

8. The method of claim 7, wherein the antibody is an anti-CD47 or an anti-EGFR antibody.

9. The method of claim 7, wherein the method promotes macrophage phagocytic activity.

10. The method of claim 7, wherein the method reduces tumor burden.

11. The method of claim 1, further comprising administering to the subject an effective amount of an immune-stimulating agent, a TLR agonist, or a checkpoint inhibitor.

12. The method of claim 1, further comprising administering to the subject an effective amount of CAR-T cells, NK cells or CAR-NK cells.

13. A pharmaceutically acceptable composition comprising human iPSC-derived macrophages.

14. The composition of claim 13, wherein the human iPSC-derived macrophages comprise a chimeric antigenic receptor (CAR) expressed thereon.

15. The composition of claim 13, wherein the CAR is Bai1, MegF10 or MerTK.
